(19) United States
(12) Patent Application Publication
(10) Pub. No.: US 2026/0184654 A1
(43) Pub. Date: Jul. 2, 2026

(54) METHODS FOR PRODUCING LIGHT OLEFINS

(71) Applicant: Dow Global Technologies LLC, Midland, MI (US)

(72) Inventors: Matthew T. Pretz, Lake Jackson, TX (US); Yang Yang, Pearland, TX (US); Liwei Li, Lake Jackson, TX (US)

(73) Assignee: Dow Global Technologies LLC, Midland, MI (US)

(21) Appl. No.: 19/131,634

(22) PCT Filed: Nov. 20, 2023
(86) PCT No.: PCT/US2023/080467

§ 371 Date: May 21, 2025

Related U.S. Application Data
(60) Provisional application No. 63/428,494, filed on Nov. 29, 2022.

(51) Int. Cl. C07C 5/333; B01J 23/90; C07C 5/48; C07C 11/04; C07C 11/06; C07C 11/08

(52) U.S. Cl. CPC C07C 5/333; B01J 23/90; C07C 5/48; C07C 11/04; C07C 11/06; C07C 11/08

(57) ABSTRACT

Methods for forming light olefins in a reactor system may include reacting a feed stream in the presence of a particulate solid in a reactor to form a product stream. The reaction may form coke on the particulate solid. The method may include passing the particulate solid to the regenerator and burning at least a portion of the coke to heat the particulate solid; mixing air and supplemental fuel upstream of the regenerator to form a gas mixture and passing the gas mixture to the regenerator through a distributor; and combusting the supplemental fuel in the regenerator to heat the particulate solid; and passing the heated particulate solid from the regenerator to the reactor. A concentration of supplemental fuel in the gas mixture may be less than 80% of the lower explosive limit of the supplemental fuel in the gas mixture, and heat from burning the coke and the supplemental fuel may be sufficient to maintain a heat balance of the reactor system.

### CROSS-REFERENCE TO RELATED APPLICATIONS

This application claims the benefit of U.S. Provisional Application Ser. No. 63/428,494 filed Nov. 29, 2022, the entire disclosure of which is hereby incorporated herein by reference.

### TECHNICAL FIELD

Embodiments described herein generally relate to chemical processing and, more specifically, to methods and systems for catalytic chemical conversion.

### BACKGROUND

Many chemicals may be produced through processes employing particulate solids, such as solid particulate catalysts. During these processes, the particulate solids may become “spent” and have reduced activity in subsequent reactions. In addition, endothermic processes require heat and the “spent” catalyst must be reheated. Thus, spent particulate solids may be transferred to a regeneration unit to be reheated and regenerated, increasing the activity of the particulate solids for use in subsequent reactions. Following regeneration in the regeneration unit, the regenerated particulate solids may be transferred back to the reactor for use in subsequent reactions.

### SUMMARY

There is a need for improved methods for regenerating particulate solids for use in the production of light olefins. Methods for regenerating particulate solids may include burning a supplemental fuel to heat the catalyst. The supplemental fuel and air may be introduced into a combustor through separate distributors. Coke may form on metal surfaces of the distributor used to inject the supplemental fuel into the combustor. The formation of coke on the distributor could plug the distributor and force a shutdown of the catalyst regeneration system. In addition, as the combustor vessel may be large and operate at extremely high temperature, the mechanical limitations of running fuel gas distributors over the entire vessel cross section may limit the size of vessel that can be constructed.

One or more of the presently disclosed methods for forming light olefins may address this problem. In one or more embodiments, air and supplemental fuel may be mixed upstream of the combustor. This gas mixture may be injected into the combustor through a single distributor, which may reduce the formation of coke on the distributor and may eliminate the need to run distributors across the vessel cross section for vessels operating at temperatures from 750° C. to 915° C. The concentration of supplemental fuel and air in the gas mixture may be controlled such that the percentage of supplemental fuel in the gas mixture is less than the lower explosive limit of the supplemental fuel. This may reduce the risk of combustion of the gas mixture before the gas mixture is injected into the combustor.

According to one or more embodiments disclosed herein, a method for forming light olefins in a reactor system comprising reactor and a regenerator may comprise reacting a feed stream in the presence of a particulate solid in the reactor to form a product stream. The reaction may form coke on the particulate solid, and the reaction may be an endothermic reaction. The method may comprise passing the particulate solid to the regenerator and burning at least a portion of the coke to heat the particulate solid. The method may include mixing air and supplemental fuel upstream of the regenerator to form a gas mixture and passing the gas mixture to the regenerator through a distributor. The method may include combusting the supplemental fuel in the regenerator to heat the particulate solid and passing the heated particulate solid from the regenerator to the reactor. A concentration of supplemental fuel in the gas mixture may be less than 80% of the lower explosive limit of the supplemental fuel in the gas mixture, and heat from burning the at least a portion of the coke and the supplemental fuel may be sufficient to maintain a heat balance of the reactor system.

### BRIEF DESCRIPTION OF THE DRAWINGS

The following detailed description of specific embodiments of the present disclosure can be best understood when read in conjunction with the following drawings, where like structure is indicated with like reference numerals and in which:

FIG. 1 schematically depicts a reactor system comprising a reactor section and a regenerator section, according to one or more embodiments disclosed herein; and

FIG. 2 schematically depicts a cross-sectional view of a plate grid distributor, according to one or more embodiments disclosed herein.

It should be understood that the drawings are schematic in nature, and do not include some components of a fluid catalytic reactor system commonly employed in the art, such as, without limitation, temperature transmitters, pressure transmitters, flow meters, pumps, valves, and the like. It would be known that these components are within the spirit and scope of the present embodiments disclosed. However, operational components, such as those described in the present disclosure, may be added to the embodiments described in this disclosure.

Reference will now be made in greater detail to various embodiments, some embodiments of which are illustrated in the accompanying drawings. Whenever possible, the same reference numerals will be used throughout the drawings to refer to the same or similar parts.

### DETAILED DESCRIPTION

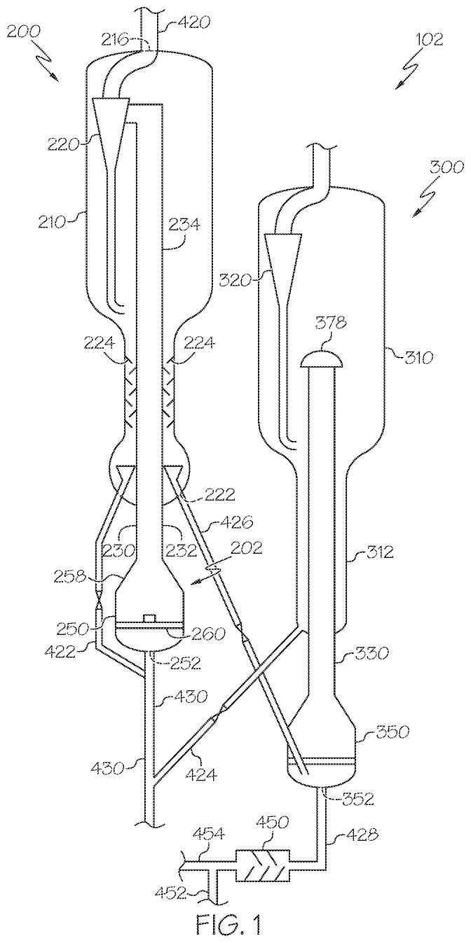

As described herein, methods for producing light olefins may include reacting a feed stream in the presence of a particulate solid to form a product stream. The reaction may form coke on the particulate solid, and the particulate solid may be regenerated by burning the coke to heat the particulate solid. The method may also include mixing supplemental fuel and air to form a gas mixture, passing the gas mixture to the regenerator, and burning the supplemental fuel in the regenerator to heat the particulate solid. The heated particulate solid may be passed back to the reactor. Such methods may utilize systems that have particular features, such as a particular orientation of system parts. One particular embodiment, which is disclosed in detail herein, is depicted in FIG. 1. However, it should be understood that the principles disclosed and taught herein may be applicable to other systems which utilize different system components oriented in different ways, or different reaction schemes utilizing various catalyst compositions.

In non-limiting examples, the reactor system 102 described herein may be utilized to produce light olefins from hydrocarbon feed streams. Light olefins may be produced from a variety of hydrocarbon feed streams by utilizing different reaction mechanisms. For example, light olefins may be produced by at least dehydrogenation reactions, cracking reactions, dehydration reactions, and methanol-to-olefin reactions. These reaction types may utilize different feed streams and different particulate solids to produce light olefins. It should be understood that when “particulate solids” are referred to herein, they may equally refer to the catalyst referenced with respect to the system of FIG. 1.

Now referring to FIG. 1, an example reactor system 102 which may be suitable for use with the methods described herein is schematically depicted. However, it should be understood that other reactor system configurations may be suitable for the methods described herein. The reactor system 102 generally comprises multiple system components, such as a reactor portion 200 and/or a catalyst processing portion 300. As used herein in the context of FIG. 1, the reactor portion 200 generally refers to the portion of a reactor system 102 in which the major process reaction takes place. The reactor portion 200 comprises a reactor 202, which may include a downstream reactor section 230 and an upstream reactor section 250. According to one or more embodiments, as depicted in FIG. 1, the reactor portion 200 may additionally include a catalyst separation section 210, which serves to separate the catalyst from the chemical products formed in the reactor 202. Also, as used herein, the catalyst processing portion 300 generally refers to the portion of a reactor system 102 where the catalyst is in some way processed, such as by combustion. The catalyst processing portion 300 may comprise a combustor 350 and a riser 330, and may optionally comprise a catalyst separation section 310. In some embodiments, the catalyst may be regenerated by burning off contaminants like coke in the catalyst processing portion 300. In embodiments, the catalyst may be heated in the catalyst processing portion 300. A supplemental fuel may be utilized to heat the catalyst in the catalyst processing portion 300 if coke or another combustible material is not formed on the catalyst, or an amount of coke formed on the catalyst is not sufficient to burn off to heat the catalyst to a desired temperature. In one or more embodiments, the catalyst separation section 210 may be in fluid communication with the combustor 350 (e.g., via standpipe 426) and the catalyst separation section 310 may be in fluid communication with the upstream reactor section 250 (e.g., via standpipe 424 and transport riser 430).

As described with respect to FIG. 1, the feed stream may enter transport riser 430, and the product stream may exit the reactor system 102 via pipe 420. According to one or more embodiments, the reactor system 102 may be operated by feeding a chemical feed (e.g., in a feed stream) and a fluidized catalyst into the upstream reactor section 250. The chemical feed contacts the catalyst in the upstream reactor section 250, and each flow upwardly into and through the downstream reactor section 230 to produce a chemical product. The chemical product and the catalyst may be passed out of the downstream reactor section 230 to a separation device 220 in the catalyst separation section 210, where the catalyst is separated from the chemical product, which is transported out of the catalyst separation section 210. The separated catalyst is passed from the catalyst separation section 210 to the combustor 350. In the combustor 350, the catalyst may be processed by, for example, combustion. For example, and without limitation, the catalyst may be de-coked and/or supplemental fuel may be combusted to heat the catalyst. The catalyst is then passed out of the combustor 350 and through the riser 330 to a riser termination separator 378, where the gas and solid components from the riser 330 are at least partially separated. The vapor and remaining solids are transported to a secondary separation device 320 in the catalyst separation section 310 where the remaining catalyst is separated from the gases from the catalyst processing (e.g., gases emitted by combustion of spent catalyst or supplemental fuel). The separated catalyst is then passed from the catalyst separation section 310 to the upstream reactor section 250 via standpipe 424 and transport riser 430, where it is further utilized in a catalytic reaction. Thus, the catalyst, in operation, may cycle between the reactor portion 200 and the catalyst processing portion 300. In general, the processed chemical streams, including the feed streams and product streams may be gaseous, and the catalyst may be fluidized particulate solid.

Additionally, as described herein, the structural features of the reactor section 200 and regeneration section 300 may be similar or identical in some respects. For example, each of the reactor section 200 and regeneration section 300 include a reaction vessel (i.e., upstream reactor section 250 of the reactor section 200 and combustor 350 of the regeneration section 300), a riser (i.e., riser 230 of the reactor section 200 and riser 330 of the regeneration section 300), and a particulate solid separation section (i.e., particulate solid separation section 210 of the reactor section 200 and particulate solid separation section 310 of the regeneration section 300). It should be appreciated that since many of the structural features of the reactor section 200 and the regeneration section 300 may be similar or identical in some respects, similar or identical portions of the reactor section 200 and the regeneration section 300 have been provided reference numbers throughout this disclosure with the same final two digits, and disclosures related to one portion of the reactor section 200 may be applicable to the similar or identical portion of the regeneration section 300, and vice versa.

According to one or more embodiments described herein, the reactor portion 200 may comprise an upstream reactor section 250, a transition section 258, and a downstream reactor section 230, such as a riser. The transition section 258 may connect the upstream reactor section 250 with the downstream reactor section 230. According to one or more embodiments, the upstream reactor section 250 and the downstream reactor section 230 may each have a substantially constant cross-section area, while the transition section 258 may be tapered and does not have a constant cross-sectional area. As described herein, unless otherwise explicitly stated, the “cross-sectional area” refers to the area of the cross section of a portion of the reactor part in a plane substantially orthogonal to the direction of general flow of reactants and/or products. For example, in FIG. 1, the cross sectional area of the upstream reactor section 250, the transition section 258, and the downstream reactor section 230 is in the direction of a plane defined by the horizontal direction and the direction into the page (orthogonal to the direction of fluid motion, i.e., vertically upward in FIG. 1).

As depicted in FIG. 1, the upstream reactor section 250 may be positioned below the downstream reactor section 230. Such a configuration may be referred to as an upflow configuration in the reactor 202.

As described herein, the upstream reactor section 250 may include a vessel, drum, barrel, vat, or other container suitable for a given chemical reaction. In one or more embodiments, the upstream reactor section 250 may be generally cylindrical in shape (i.e., having a substantially circular cross-sectional shape), or may alternately be non-cylindrically shaped, such as prism shaped with cross-sectional shapes of triangles, rectangles, pentagons, hexagons, octagons, ovals, or other polygons or curved closed shapes, or combinations thereof. The upstream reactor section 250, as used throughout this disclosure, may generally include a metallic frame, and may additionally include refractory linings or other materials utilized to protect the metallic frame and/or control process conditions. As depicted in FIG. 1, the upstream reactor section 250 may include a lower reactor portion catalyst inlet port 252 defining the connection of transport riser 430 to the upstream reactor section 250.

It should be understood that any two quantitative values assigned to a property may constitute a range of that property, and all combinations of ranges formed from all stated quantitative values of a given property are contemplated in this disclosure. It should be appreciated that compositional ranges of a chemical constituent in a composition should be appreciated as containing, in some embodiments, a mixture of isomers of that constituent. In additional embodiments, the chemical compounds may be present in alternative forms such as derivatives, salts, hydroxides, etc. Generally, “inlet ports” and “outlet ports” of any system unit of the reactor system 102 described herein refer to openings, holes, channels, apertures, gaps, or other like mechanical features in the system unit. For example, inlet ports allow for the entrance of materials to the particular system unit and outlet ports allow for the exit of materials from the particular system unit. Generally, an outlet port or inlet port will define the area of a system unit of the reactor system 102 to which a pipe, conduit, tube, hose, transport line, or like mechanical feature is attached, or to a portion of the system unit to which another system unit is directly attached. While inlet ports and outlet ports may sometimes be described herein functionally in operation, they may have similar or identical physical characteristics, and their respective functions in an operational system should not be construed as limiting on their physical structures.

The upstream reactor section 250 may be connected to a transport riser 430, which, in operation, may provide processed catalyst and/or reactant chemicals in a feed stream to the reactor portion 200. The processed catalyst and/or reactant chemicals may be mixed with a distributor 260 housed in the upstream reactor section 250. The catalyst entering the upstream reactor section 250 via transport riser 430 may be passed through standpipe 424 to a transport riser 430, thus arriving from the catalyst processing portion 300. In some embodiments, catalyst may come directly from the catalyst separation section 210 via standpipe 422 and into a transport riser 430, where it enters the upstream reactor section 250. The catalyst can also be fed via 422 directly to the upstream reactor section 250. This catalyst may be slightly deactivated, but may still, in some embodiments, be suitable for reaction in the upstream reactor section 250. As used herein, “deactivated” may refer to a catalyst which is contaminated with a substance such as coke, or is cooler in temperature than desired. Regeneration may remove the contaminant such as coke, raise the temperature of the catalyst, or both.

Still referring to FIG. 1, the reactor portion 200 may comprise a downstream reactor section 230 which acts to transport reactants, products, and/or catalyst from the upstream reactor section 250 to the catalyst separation section 210. In one or more embodiments, the downstream reactor section 230 may be generally cylindrical in shape (i.e., having a substantially circular cross-sectional shape), or may alternately be non-cylindrically shaped, such as prism shaped with cross-sectional shape of triangles, rectangles, pentagons, hexagons, octagons, ovals, or other polygons or curved closed shapes, or combinations thereof. The downstream reactor section 230, as used throughout this disclosure, may generally include a metallic frame, and may additionally include refractory linings or other materials utilized to protect the metallic frame and/or control process conditions.

According to some embodiments, the downstream reactor section 230 may include an external riser section 232 and an internal riser section 234. As used herein, an “external riser section” refers to the portion of the riser that is outside of the catalyst separation section, and an “internal riser section” refers to the portion of the riser that is within the catalyst separation section. For example, in the embodiment depicted in FIG. 1, the internal riser section 234 of the reactor portion 200 may be positioned within the catalyst separation section 210, while the external riser section 232 is positioned outside of the catalyst separation section 210.

As depicted in FIG. 1, the upstream reactor section 250 may be connected to the downstream reactor section 230 via the transition section 258. The upstream reactor section 250 may generally comprise a greater cross-sectional area than the downstream reactor section 230. The transition section 258 may be tapered from the size of the cross-section of the upstream reactor section 250 to the size of the cross-section of the downstream reactor section 230 such that the transition section 258 projects inwardly from the upstream reactor section 250 to the downstream reactor section 230.

In some embodiments, such as those where the upstream reactor section 250 and the downstream reactor section 230 have similar cross-sectional shapes, the transition section 258 may be shaped as a frustum. For example, for an embodiment of a reactor portion 200 comprising a cylindrical upstream reactor section 250 and cylindrical downstream reactor section 230, the transition section 258 may be shaped as a conical frustum. However, it should be understood that a wide variety of upstream reactor section 250 shapes are contemplated herein which connect various shapes and sizes of upstream reactor section 250 and downstream reactor section 230.

In operation, the catalyst may move upward through the downstream reactor section 230 (from the upstream reactor section 250), and into the separation device 220. The separated vapors may be removed from the reactor system 102 via a pipe 420 at a gas outlet port 216 of the catalyst separation section 210. According to one or more embodiments, the separation device 220 may be a cyclonic separation system, which may include two or more stages of cyclonic separation. In embodiments where the separation device 220 comprises more than one cyclonic separation stages, the first separation device into which the fluidized stream enters is referred to a primary cyclonic separation device. The fluidized effluent from the primary cyclonic separation device may enter into a secondary cyclonic separation device for further separation. Primary cyclonic separation devices may include, for example, primary cyclones, and systems commercially available under the names VSS (commercially available from UOP), LD2 (commercially available from Stone and Webster), and RS2 (commercially available from Stone and Webster). Primary cyclones are described, for example, in U.S. Pat. Nos. 4,579,716; 5,190,650; and 5,275,641, which are each incorporated by reference in their entirety herein. In some separation systems utilizing primary cyclones as the primary cyclonic separation device, one or more set of additional cyclones, e.g. secondary cyclones and tertiary cyclones, are employed for further separation of the catalyst from the product gas. It should be understood that any primary cyclonic separation device may be used in embodiments of the invention.

According to one or more embodiments, following separation from vapors in the separation device 220, the catalyst may generally move through the stripper 224 to the catalyst outlet port 222 where the catalyst is transferred out of the reactor portion 200 via standpipe 426 and into the catalyst processing portion 300. Optionally, the catalyst may also be transferred directly back into the upstream reactor section 250 via standpipe 422. Alternatively, the catalyst may be premixed with processed catalyst in the transport riser 430.

As is described in detail in accordance with the embodiment of FIG. 1, according to one or more embodiments, the catalyst may be processed by one or more of the steps of passing the catalyst from the reactor 202 to the combustor 350, burning a supplemental fuel source in the combustor 350 to heat the catalyst, and passing the heated catalyst from the combustor 350 to the reactor 202.

Referring now to the catalyst processing portion 300, as depicted in FIG. 1, the combustor 350 of the catalyst processing portion 300 may include one or more lower reactor portion inlet ports 352 and may be in fluid communication with the riser 330. The combustor 350 may be in fluid communication with the catalyst separation section 210 via standpipe 426, which may supply spent catalyst from the reactor portion 200 to the catalyst processing portion 300 for regeneration.

In one or more embodiments, a gas mixture comprising air and supplemental fuel may be passed to the combustor 350 through the lower reactor inlet port 352. The gas mixture may be formed by mixing the air and supplemental fuel upstream of the combustor 350. In embodiments, the air and supplemental fuel may be mixed in static mixer 450. Static mixer 450 may be any static mixer suitable for mixing gasses. For example, the static mixer may comprise a housing and one or more baffles positioned within the housing. In embodiments, the one or more baffles may be helically shaped. In embodiments, the static mixer may be a plate style static mixer. Without intending to be bound by theory, it is believed that the structure of the static mixer creates turbulent flow, which mixes the fluid flowing through the static mixer. The gas mixture may be passed from the static mixer 450 to the combustor 350 through conduit 428. In embodiments, air may be passed to the static mixer 450 through conduit 452 and supplemental fuel may be passed to the static mixer 450 through conduit 454. In one or more embodiments, the supplemental fuel may comprise hydrogen, methane, natural gas, ethane, propane, or any gas that produces heat upon combustion. In one or more embodiments, the air may be enriched in oxygen. For example, the enriched air may comprise greater than 21 mol. % oxygen or from 21 mol. % oxygen to 40 mol. % oxygen.

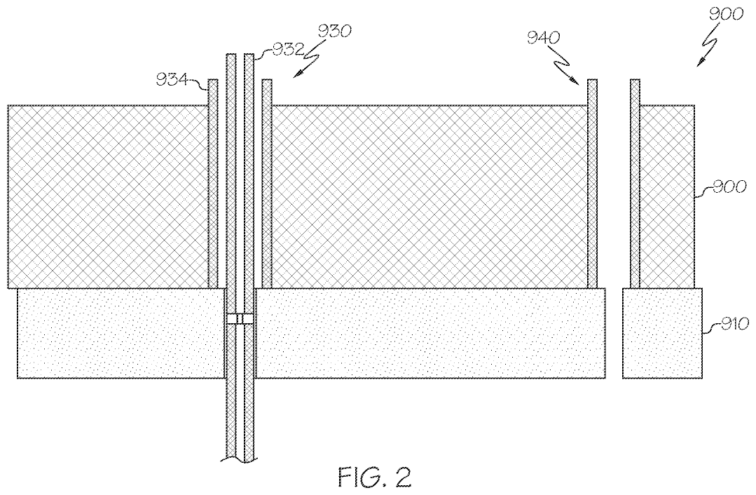

In one or more embodiments, a fuel gas distributor may inject the supplemental fuel into the air upstream of the static mixer in conduit 452. Without intending to be bound by theory, this may result in more even distribution of the supplemental fuel in the air when the air and supplemental fuel are passed to the static mixer 450. Referring now to FIG. 2, the fuel gas distributor may be a plate grid distributor 900. In embodiments, the plate grid distributor 900 may comprise a plate 910 and a refractory lining 920 on the downstream side of the plate 910. The plate grid distributor 900 may comprise multiple injection points (injection points 930 and 940 depicted in FIG. 2). In the embodiment depicted in FIG. 2, injection point 930 is configured to pass both air and supplemental fuel through the injection point 930. Supplemental fuel may pass through tube 932, and air may pass through an annular space between tube 934 and tube 932 and between the plate 910 and tube 932. It should be understood that tube 932 may be connected to a supplemental fuel source (not depicted). In the embodiment depicted in FIG. 2, injection point 940 is configured to pass air through the injection point 940.

In some embodiments, fuel gas and air may be passed through each injection point of the distributor. In some embodiments, fuel gas and air may be individually passed through separate injections points. In one or more embodiments, there may be at an air injection point for each fuel gas injection. For example, there may be more than one air injection point for each fuel gas injection point. For example, there may be from 2 to 10 air injection point for each fuel gas injection point. Without intending to be bound by theory, passing fuel gas and air through each injection point may result in good fuel gas distribution, but may be a relatively complex system to design and maintain. Likewise, a distributor including a fuel gas injection point for each air injection point may provide good fuel gas distribution, but be a relatively complex system to design and maintain. On the other hand, including multiple air injection points for each fuel gas injection point may reduce the complexity of the fuel gas distributor while maintaining satisfactory fuel gas distribution.

Without intending to be bound by theory, and referring again to FIG. 1, mixing the air and the supplemental fuel upstream of the combustor 350 may result in a more homogeneous mixture of supplemental fuel and air in the combustor 350 than would occur by introducing supplemental fuel and introducing air separately into the combustor. A homogeneous mixture of air and supplemental fuel may ensure that the stoichiometric ratio of oxygen to fuel locally is sufficient for the fuel to combust once the fuel enters the combustor 350. A homogenous mixture of air and supplemental fuel may contribute to uniform combustion of the fuel within the combustor 350, which could reduce the formation of hot spots within the combustor 350. Additionally, if the air and supplemental fuel are not mixed homogeneously, then there may be local areas where the lower explosive limit of the fuel is exceeded.

In embodiments, the gas mixture may be introduced to the combustor 350 through a single distributor. The distributor may comprise a plurality of nozzles operable to uniformly distribute the gas mixture in the combustor 350. Suitable fuel gas distributors are described in U.S. Pat. No. 9,889,418, the entirety of which is incorporated by reference herein. Without intending to be bound by theory, introducing the gas mixture through a single distributor may reduce the likelihood of coke building up on the distributor. When supplemental fuel and air are introduced into the combustor 350 individually, each through its own distributor, coke may form on the fuel gas distributor under the high temperature conditions of the combustor 350. Such coke accumulation may plug the distributor, which may result in uneven introduction of supplemental fuel into the combustor 350 and may even lead to a forced shutdown of the reactor system 102. Without intending to be bound by theory, when a plate grid distributor is used, a thermal insulating refractory may be installed on the top of the distributor to control the mixture of air and supplemental fuel to a desired temperature. The refractory may insulate the bottom of the distributor so the bottom of the distributor is the same temperature of the gasses flowing through the distributor. The pipes flowing to the distributor may be at least partially covered by a thin, high-density refractory material, which may be a less effective insulator than the refractory installed on the top of the distributor.

In one or more embodiments, the concentration by volume of supplemental fuel in the gas mixture when fully mixed may be below the lower explosive limit of the supplemental fuel. As described herein the “lower explosive limit” (LEL) of a gas is the lowest concentration of that gas in air capable of combusting in the presence of an ignition source, such as an arc, flame, or heat. For example, the concentration of the supplemental fuel in the gas mixture may be less than or equal to 90%, less than or equal to 85%, less than or equal to 80%, less than or equal to 75%, less than or equal to 70%, less than or equal to 65%, less than or equal to 60%, less than or equal to 50%, less than or equal to 45%, less than or equal to 40% of the LEL of the supplemental fuel. In one or more embodiments, the concentration of the supplemental fuel in the gas mixture may be from 25% to 90% of the LEL. For example, the concentration of the supplemental fuel in the gas mixture may be from 25% to 90%, from 30% to 90%, from 35% to 90%, from 40% to 90%, from 45% to 90%, from 50% to 90%, from 55% to 90%, from 60% to 90%, from 65% to 90%, from 70% to 90%, from 75% to 90%, from 80% to 90%, from 85% to 90%, from 25% to 85%, from 25% to 80%, from 25% to 75%, from 25% to 70%, from 25% to 65%, from 25% to 60%, from 25% to 55%, from 25% to 50%, from 25% to 45%, from 25% to 40%, from 25% to 35%, from 25% to 30%, or any combination or sub-set of these ranges.

Generally, the LEL of a gas may change as temperature, pressure, and oxygen concentration change. For example, as temperature increases, the LEL decreases; as pressure increases, the LEL decreases; and as the concentration of oxygen increases, the LEL decreases. In one or more embodiments, the air and supplemental fuel may be mixed at ambient conditions, and the LEL of the gas mixture may be determined based on ambient conditions, such as atmospheric pressure and a temperature of about 25° C. In embodiments, the gas mixture may be injected into the combustor at about ambient temperature. In one or more embodiments, the air and supplemental fuel may be mixed at a temperature and pressure that may be greater than ambient temperature or ambient pressure or both. For example, the air and supplemental fuel may be mixed at a temperature of from ambient temperature to 400° C., from ambient temperature to 300° C., from ambient temperature to 200° C., or from ambient temperature to 100° C. In such embodiments, the LEL at the mixing conditions may be predicted mathematically. The LEL may be predicted and controlled by a control system including sensors, such as a temperature sensor, a pressure sensor, and an analyzers that could measure the concentration of fuel gas or oxygen, The control system may be able to automatically adjust the flow of air and fuel gas to prevent the mixture of air and fuel gas from exceeding a LEL target.

In one or more embodiments, the gas mixture may be heated between the static mixer 450 and the combustor 350. For example, the gas mixture may be heated before the gas mixture is passed to the regenerator through the distributor. In such embodiments, the concentration of the supplemental fuel in the gas mixture may be sufficiently below the LEL at ambient conditions that the concentration of supplemental fuel in the gas mixture may remain below the LEL once the gas mixture has been heated. In one or more embodiments, the gas mixture may be heated to a temperature of up to and including 800° C. For example, the gas mixture may be heated to a temperature of up to and including 800° C., 700° C., 600° C., 500° C., or 400° C. In embodiments, the gas mixture may be heated to a temperature of from 400° C. to 800° C., from 500° C. to 800° C., from 600° C. to 800° C., from 700° C. to 800° C., from 400 ° C. to 700° C., from 400° C. to 600° C., from 400° C. to 500° C., or any combination or subset of these ranges. In embodiments, the gas mixture may be heated to a temperature such that the concentration of the supplemental fuel is less than or equal to 80% of the LEL. For example, the gas mixture may be heated to a temperature such that the concentration of the supplemental fuel is less than or equal to 80% of the LEL, 75% of the LEL, 70% of the LEL, 65 % of the LEL, or 60% of the LEL. In embodiments, the gas mixture may be heated to a temperature less than or equal to the auto-ignition temperature of the supplemental fuel. Without intending to be bound by theory, when the gas mixture is preheated, the amount of supplemental fuel required and the amount of oxygen necessary to burn the supplemental fuel may be reduced. This may allow for smaller pieces of process equipment to be use.

Without intending to be bound by theory, when the concentration of supplemental fuel in the gas mixture is kept below the LEL of the supplemental fuel, the risk of igniting the gas mixture before the gas mixture is introduced into the combustor 350 is significantly reduced. Furthermore, the risk of explosion of the gas mixture reduced. Reducing the risk of explosion or combustion of the gas mixture is desirable because explosion or combustion of the gas mixture outside of combustor 350 may create safety issues, damage system components, or force a shutdown of the reactor system 102.

In one or more embodiments, burning the coke and the supplemental fuel in the combustor 350 may heat the catalyst. In turn, the heated catalyst may be passed to the reactor 250 and provide heat to the reactor. In one or more embodiments, the heat from burning the coke and the supplemental fuel may be sufficient to maintain a heat balance of the reactor. In one or more embodiments, burning the coke and supplemental fuel in the combustor 350 may be the sole means for heating the catalyst.

In embodiments, the light olefins may be produced by one or more endothermic reactions. As described herein, “endothermic reactions” refer to chemical processes where the enthalpy or internal energy of the system increases such that the system absorbs thermal energy from its surroundings. Without intending to be bound by theory, when an endothermic reaction occurs in the reactor 250, thermal energy may be absorbed from the catalyst entering the reactor 250 such that the catalyst exiting the reactor 250 may have a lower temperature than catalyst entering the reactor 250. Accordingly, catalyst may be heated in the combustor 350 such that the catalyst may provide sufficient heat to drive an endothermic reaction occurring in reactor 250 and maintain the heat balance of system 102.

In one or more embodiments, additional supplemental fuel may be required to heat the catalyst. In such embodiments, the reactor 350 may comprise a second distributor for injecting supplemental fuel into the combustor. The second distributor may be any suitable means for injecting supplemental fuel into the reactor.

In non-limiting examples, the reactor system 102 described herein may be utilized to produce light olefins from hydrocarbon feed streams. Light olefins may be produced from a variety of hydrocarbon feed streams by utilizing different reaction mechanisms. In one or more embodiments, the light olefins may be produced by one or more endothermic reactions. For example, light olefins may be produced by endothermic reactions including, but not limited to, dehydrogenation reactions, cracking reactions, dehydration reactions, and methanol-to-olefin reactions. These reaction types may utilize different feed streams and different particulate solids to produce light olefins. It should be understood that when “catalysts” are referred to herein, they may equally refer to the particulate solid referenced with respect to the system of FIG. 1.

According to one or more embodiments, the reaction may be a dehydrogenation reaction. According to such embodiments, the hydrocarbon feed stream may comprise one or more of ethyl benzene, ethane, propane, n-butane, and i-butane. In one or more embodiments, the hydrocarbon feed stream may comprise at least 50 wt. %, at least 60 wt. %, at least 70 wt. %, at least 80 wt. %, at least 90 wt. %, at least 95 wt. % or even at least 99 wt. % of ethyl benzene. In one or more embodiments, the hydrocarbon feed stream may comprise at least 50 wt. %, at least 60 wt. %, at least 70 wt. %, at least 80 wt. %, at least 90 wt. %, at least 95 wt. % or even at least 99 wt. % of ethane. In additional embodiments, the hydrocarbon feed stream may comprise at least 50 wt. %, at least 60 wt. %, at least 70 wt. %, at least 80 wt. %, at least 90 wt. %, at least 95 wt. % or even at least 99 wt. % of propane. In additional embodiments, the hydrocarbon feed stream may comprise at least 50 wt. %, at least 60 wt. %, at least 70 wt. %, at least 80 wt. %, at least 90 wt. %, at least 95 wt. % or even at least 99 wt. % of n-butane. In additional embodiments, the hydrocarbon feed stream may comprise at least 50 wt. %, at least 60 wt. %, at least 70 wt. %, at least 80 wt. %, at least 90 wt. %, at least 95 wt. % or even at least 99 wt. % of i-butane. In additional embodiments, the hydrocarbon feed stream may comprise at least 50 wt. %, at least 60 wt. %, at least 70 wt. %, at least 80 wt. %, at least 90 wt. %, at least 95 wt. % or even at least 99 wt. % of the sum of ethane, propane, n-butane, and i-butane.

In one or more embodiments, the dehydrogenation reaction may utilize gallium and/or platinum particulate solids as a catalyst. In such embodiments, the particulate solids may comprise a gallium and/or platinum catalyst. As described herein, a gallium and/or platinum catalyst comprises gallium, platinum, or both. The gallium and/or platinum catalyst may be carried by an alumina or alumina silica support, and may optionally comprise potassium. Such gallium and/or platinum catalysts are disclosed in U.S. Pat. No. 8,669,406, which is incorporated herein by reference in its entirety. However, it should be understood that other suitable catalysts may be utilized to perform the dehydrogenation reaction.

In one or more embodiments, the reaction mechanism may be dehydrogenation followed by combustion (in the same chamber). In such embodiments, a dehydrogenation reaction may produce hydrogen as a byproduct, and an oxygen carrier material may contact the hydrogen and promote combustion of the hydrogen, forming water. The oxygen carrier material may include one or more transition metal oxides. According to one or more embodiments, the one or more transition metal oxides may be a redox-active transition metal oxide. The redox-active transition metal oxide includes binary, ternary, or other mixed metal oxides capable of undergoing reduction in the presence of a reducing agent (for example, hydrogen) and oxidation in the presence of oxidizing agent (for example, oxygen or air). In some embodiments the redox-active transition metal oxide may be chosen from Mn₂O₃, Fe₂O₃, Co₃O₄, CuO, (LaSr)CoO₃, (LaSr)MnO₃, Mg₆MnO₈, MgMnO₃, MnO₂, Fe₃O₄, Mn₃O₄, and Cu₂O. In some embodiments, the oxygen carrier material may be a solid. In specific embodiments, the oxygen carrier material may be a crushed solid or powder. Examples of such reaction mechanisms, which are contemplated as possible reactions mechanisms for the systems and methods described herein, are disclosed in WO 2020/046978 and WO 2019/048391, the teachings of which are incorporated by reference in their entirety herein.

According to one or more embodiments, the reaction may be a cracking reaction. According to such embodiments, the hydrocarbon feed stream may comprise one or more of naphtha, n-butane, or i-butane. According to one or more embodiments, the hydrocarbon feed stream may comprise at least 50 wt. %, at least 60 wt. %, at least 70 wt. %, at least 80 wt. %, at least 90 wt. %, at least 95 wt. % or even at least 99 wt. % of naphtha. In additional embodiments, the hydrocarbon feed stream may comprise at least 50 wt. %, at least 60 wt. %, at least 70 wt. %, at least 80 wt. %, at least 90 wt. %, at least 95 wt. % or even at least 99 wt. % of n-butane. In additional embodiments, the hydrocarbon feed stream may comprise at least 50 wt. %, at least 60 wt. %, at least 70 wt. %, at least 80 wt. %, at least 90 wt. %, at least 95 wt. % or even at least 99 wt. % of i-butane. In additional embodiments, the hydrocarbon feed stream may comprise at least 50 wt. %, at least 60 wt. %, at least 70 wt. %, at least 80 wt. %, at least 90 wt. %, at least 95 wt. % or even at least 99 wt. % of the sum of naphtha, n-butane, and i-butane.

In one or more embodiments, the cracking reaction may utilize one or more zeolites as a catalyst. In such embodiments, the particulate solids may comprise one or more zeolites. In some embodiments, the one or more zeolites utilized in the cracking reaction may comprise a ZSM-5 zeolite. However, it should be understood that other suitable catalysts may be utilized to perform the cracking reaction. For example, suitable catalysts that are commercially available may include Intercat Super Z Excel or Intercat Super Z Exceed. In additional embodiments, the cracking catalyst may comprise, in addition to a catalytically active material, platinum. For example, the cracking catalyst may include from 0.001 wt. % to 0.05 wt. % of platinum. The platinum may be sprayed on as platinum nitrate and calcined at an elevated temperature, such as around 700° C. Without being bound by theory, it is believed that the addition of platinum to the catalyst may allow for easier combustion of supplemental fuels, such as methane.

According to one or more embodiments, the reaction may be a dehydration reaction. According to such embodiments, the hydrocarbon feed stream may comprise one or more of ethanol, propanol, or butanol. According to one or more embodiments, the hydrocarbon feed stream may comprise at least 50 wt. %, at least 60 wt. %, at least 70 wt. %, at least 80 wt. %, at least 90 wt. %, at least 95 wt. % or even at least 99 wt. % of ethanol. In additional embodiments, the hydrocarbon feed stream may comprise at least 50 wt. %, at least 60 wt. %, at least 70 wt. %, at least 80 wt. %, at least 90 wt. %, at least 95 wt. % or even at least 99 wt. % of propanol. In additional embodiments, the hydrocarbon feed stream may comprise at least 50 wt. %, at least 60 wt. %, at least 70 wt. %, at least 80 wt. %, at least 90 wt. %, at least 95 wt. % or even at least 99 wt. % of butanol. In additional embodiments, the hydrocarbon feed stream or may comprise at least 50 wt. %, at least 60 wt. %, at least 70 wt. %, at least 80 wt. %, at least 90 wt. %, at least 95 wt. % or even at least 99 wt. % of the sum of ethanol, propanol, and butanol.

In one or more embodiments, the dehydration reaction may utilize one or more acid catalysts. In such embodiments, the particulate solids may comprise one or more acid catalysts. In some embodiments, the one or more acid catalysts utilized in the dehydration reaction may comprise a zeolite (such as ZSM-5 zeolite), alumina, amorphous aluminosilicate, acid clay, or combinations thereof. For example, commercially available alumina catalysts which may be suitable, according to one or more embodiments, include SynDol (available from Scientific Design Company), V200 (available from UOP), or P200 (available from Sasol). Commercially available zeolite catalysts which may be suitable include CBV 8014, CBV 28014 (each available from Zeolyst). Commercially available amorphous aluminosilicate catalysts which may be suitable include silica-alumina catalyst support, grade 135 (available from Sigma Aldrich). However, it should be understood that other suitable catalysts may be utilized to perform the dehydration reaction.

According to one or more embodiments, the reaction may be a methanol-to-olefin reaction. According to such embodiments, the hydrocarbon feed stream may comprise methanol. According to one or more embodiments, the hydrocarbon feed stream may comprise at least 50 wt. %, at least 60 wt. %, at least 70 wt. %, at least 80 wt. %, at least 90 wt. %, at least 95 wt. % or even at least 99 wt. % of methanol.

In one or more embodiments, the methanol-to-olefin reaction may utilize one or more zeolites as a catalyst. In such embodiments, the particulate solids may comprise one or more zeolites. In some embodiments, the one or more zeolites utilized in the methanol-to-olefin reaction may comprise a one or more of a ZSM-5 zeolite or a SAPO-34 zeolite. However, it should be understood that other suitable catalysts may be utilized to perform the methanol-to-olefin reaction.

In one or more embodiments, the operating of chemical process may include passing the product stream out of the reactor. The product stream may comprise light olefins or alkyl aromatic olefins, such as styrene. As described herein, “light olefins” refers to one or more of ethylene, propylene, or butene. As described herein, butene many include any isomer of butene, such as a-butylene, cis-β-butylene, trans-β-butylene, and isobutylene. In one embodiment, the product stream may comprise at least 30 wt. % light olefins. For example, the product stream may comprise at least 30 wt. % light olefins, at least 40 wt. % light olefins, at least 50 wt. % light olefins, at least 60 wt. % light olefins, at least 70 wt. % light olefins, at least 80 wt. % light olefins, at least 90 wt. % light olefins, at least 95 wt. % light olefins, or even at least 99 wt. % light olefins.

It is noted that one or more of the following claims utilize the term “wherein” as a transitional phrase. For the purposes of defining the present technology, it is noted that this term is introduced in the claims as an open-ended transitional phrase that is used to introduce a recitation of a series of characteristics of the structure and should be interpreted in like manner as the more commonly used open-ended preamble term “comprising.”

It should be understood that where a first component is described as “comprising” a second component, it is contemplated that, in some embodiments, the first component “consists” or “consists essentially of” that second component. It should further be understood that where a first component is described as “comprising” a second component, it is contemplated that, in some embodiments, the first component comprises at least 10%, at least 20%, at least 30%, at least 40%, at least 50%, at least 60%, at least 70%, at least 80%, at least 90%, at least 95%, or even at least 99% that second component (where % can be weight % or molar %).

According to a first aspect of the present disclosure, method for forming light olefins in a reactor system comprising a reactor and a regenerator comprises: reacting a feed stream in the presence of a particulate solid in the reactor to form a product stream, wherein the reaction forms coke on the particulate solid, and wherein the reaction is an endothermic reaction; passing the particulate solid to the regenerator and burning at least a portion of the coke to heat the particulate solid; mixing air and supplemental fuel upstream of the regenerator to form a gas mixture and passing the gas mixture to the regenerator through a distributor; combusting the supplemental fuel in the regenerator to heat the particulate solid; and passing the heated particulate solid from the regenerator to the reactor. A concentration of supplemental fuel in the gas mixture is less than 80% of the lower explosive limit of the supplemental fuel in the gas mixture. The heat from burning the at least a portion of the coke and the supplemental fuel is sufficient to maintain a heat balance of the reactor system.

A second aspect of the present disclosure may include the first aspect, wherein the supplemental fuel comprises hydrogen, methane, ethane, propane, or natural gas.

A third aspect of the present disclosure may include either the first or second aspect, wherein the concentration of supplemental fuel in the gas mixture is from 25% of the lower explosive limit to 70% of the lower explosive limit.

A fourth aspect of the present disclosure may include any of the first through third aspects, wherein mixing the air and the supplemental fuel comprises passing the air and the supplemental fuel through a static mixer.

A fifth aspect of the present disclosure may include any of the first through fourth aspects, wherein mixing the air and the supplemental fuel comprises injecting the supplemental fuel into the air through a fuel gas distributor.

A sixth aspect of the present disclosure may include any of the first through fifth aspects, wherein mixing the air and the supplemental fuel occurs at a temperature of from ambient temperature to 400° C.

A seventh aspect of the present disclosure may include any of the first through sixth aspects, wherein the method further comprises heating the gas mixture before passing the gas mixture to the regenerator through the distributor.

An eighth aspect of the present disclosure may include any of the first through seventh aspects, wherein reacting the feed stream comprises a dehydrogenation reaction and a hydrogen combustion reaction.

A ninth aspect of the present disclosure may include any of the first through seventh aspects, wherein reacting the feed stream comprises a cracking reaction.

A tenth aspect of the present disclosure may include any of the first through seventh aspects, wherein reacting the feed stream comprises a dehydration reaction.

An eleventh aspect of the present disclosure may include any of the first through seventh aspects, wherein reacting the feed stream comprises a methanol-to-olefin reaction.

A twelfth aspect of the present disclosure may include any of the first through eleventh aspects, wherein the product stream comprises one or more of ethylene, propylene, or butene.

A thirteenth aspect of the present disclosure may include any of the first through twelfth aspects, wherein the product stream comprises at least 30 wt. % light olefins.

Additionally, the term “consisting essentially of” is used in this disclosure to refer to quantitative values that do not materially affect the basic and novel characteristic(s) of the disclosure. For example, a chemical composition “consisting essentially” of a particular chemical constituent or group of chemical constituents should be understood to mean that the composition includes at least about 99.5% of a that particular chemical constituent or group of chemical constituents.

The subject matter of the present disclosure has been described in detail and by reference to specific embodiments. It should be understood that any detailed description of a component or feature of an embodiment does not necessarily imply that the component or feature is essential to the particular embodiment or to any other embodiment. Further, it should be apparent to those skilled in the art that various modifications and variations can be made to the described embodiments without departing from the spirit and scope of the claimed subject matter.

1. A method for forming light olefins in a reactor system comprising a reactor and a regenerator, the method comprising:
reacting a feed stream in the presence of a particulate solid in the reactor to form a product stream, wherein the reaction forms coke on the particulate solid, and wherein the reaction is an endothermic reaction;
passing the particulate solid to the regenerator and burning at least a portion of the coke to heat the particulate solid;
mixing air and supplemental fuel upstream of the regenerator to form a gas mixture and passing the gas mixture to the regenerator through a distributor;
combusting the supplemental fuel in the regenerator to heat the particulate solid; and
passing the heated particulate solid from the regenerator to the reactor,
wherein:
a concentration of supplemental fuel in the gas mixture is less than 80% of the lower explosive limit of the supplemental fuel in the gas mixture; and
the heat from burning the at least a portion of the coke and the supplemental fuel is sufficient to maintain a heat balance of the reactor system.

2. The method of claim 1, wherein the supplemental fuel comprises hydrogen, methane, ethane, propane, or natural gas.

3. The method of claim 1, wherein the concentration of supplemental fuel in the gas mixture is from 25% of the lower explosive limit to 70% of the lower explosive limit.

4. The method of claim 1, wherein mixing the air and the supplemental fuel comprises passing the air and the supplemental fuel through a static mixer.

5. The method of claim 1, wherein mixing the air and the supplemental fuel comprises injecting the supplemental fuel into the air through a fuel gas distributor.

6. The method of claim 1, wherein mixing the air and the supplemental fuel occurs at a temperature of from ambient temperature to 400° C.

7. The method of any of claim 1, wherein the method further comprises heating the gas mixture before passing the gas mixture to the regenerator through the distributor.

8. The method of claim 1, wherein reacting the feed stream comprises a dehydrogenation reaction and a hydrogen combustion reaction.

9. The method of claim 1, wherein reacting the feed stream comprises a cracking reaction.

10. The method of claim 1, wherein reacting the feed stream comprises a dehydration reaction.

11. The method of claim 1, wherein reacting the feed stream comprises a methanol-to-olefin reaction.

12. The method of claim 1, wherein the product stream comprises one or more of ethylene, propylene, or butene.

13. The method of claim 1, wherein the product stream comprises at least 30 wt. % light olefins.
